(19) United States
(12) Patent Application Publication
(10) Pub. No.: US 2020/0345805 A1
(43) Pub. Date: Nov. 5, 2020

(54) COMPOSITIONS AND METHODS USEABLE FOR TREATMENT OF DRY EYE

(71) Applicant: Allegro Ophthalmics, LLC, San Juan Capistrano, CA (US)

(72) Inventors: Hampar L. Karageozian, San Juan Capistrano, CA (US); John Y. Park, Santa Ana, CA (US); Vicken H. Karageozian, San Juan Capistrano, CA (US); Melvin Arbis Sarayba, Ladera Ranch, CA (US); Lisa S. Karageozian, San Juan Capistrano, CA (US); Janine M. Aubel, Laguna Niguel, CA (US)

(21) Appl. No.: 16/854,818

(22) Filed: Apr. 21, 2020

Related U.S. Application Data
(60) Provisional application No. 62/836,858, filed on Apr. 22, 2019.

(51) Int. Cl. A61K 38/08; A61K 9/00; A61K 47/18; A61K 47/02; A61K 47/38

(52) U.S. Cl. CPC A61K 38/08; A61K 9/0048; A61K 47/38; A61K 47/02; A61K 47/183

(57) ABSTRACT

Compositions and methods for treating Dry Eye in a human or non-human animal subject wherein an effective amount of a pharmaceutical composition comprising an anti-integrin peptide is administered to an eye of the subject. The anti-integrin peptide may comprise a peptide which causes at least one effect selected from: reduced expression of the Complement 3 Receptor (Integrin αMβ2); reduced leucocyte adhesion; and reduced trans-endothelial leucocyte migration. The peptide may comprises Risuteganib.

### RELATED APPLICATION

This patent application claims priority to U.S. Provisional Patent Application No. 62/836,858 entitled Compositions and Methods Useable for Treatment of Dry Eye filed Apr. 22, 2019, the entire disclosure of which is expressly incorporated herein by reference.

### FIELD OF THE INVENTION

This disclosure relates generally to the fields of chemistry, life sciences, pharmacy and medicine and more particularly to pharmaceutical preparations and their use in the treatment of eye disorders.

### BACKGROUND

Pursuant to 37 CFR 1.71(e), this patent document contains material which is subject to copyright protection and the owner of this patent document reserves all copyright rights whatsoever.

In a healthy eye, a consistent layer of tears (tear film) is distributed over the surface of the eye. This tear film keeps the eye moist and washes away dust, microbes and other debris that, if allowed to remain on the eye, could cause corneal abrasion and/or eye infection.

The term “Dry Eye” has been defined as a “multifactorial disease of the tears and ocular surface that results in symptoms of discomfort, visual disturbance, and tear film instability with potential damage to the ocular surface which is accompanied by increased osmolarity of the tear film and inflammation of the ocular surface.” See, Hessel, M., et al., Dry Eye: an Inflammatory Ocular Disease; J Ophthalmic Vis Res 2014; 9 (2): 240-250.

As used in this patent application, the term “Dry Eye” shall be interpreted to include, but is not necessarily limited to, disorders characterized in inadequate or defective tears such as those disorders referred to as: dry eye, dry eye syndrome, dry eye disease, evaporative dry eye, aqueous deficiency dry eye, keratitis sicca (dryness and inflammation of the cornea), keratoconjunctivitis sicca. (dryness that affects both the cornea and the conjunctiva) and dysfunctional tear syndrome (inadequate quality or quantity of tears).

Normal tears contain three major components, an oily (lipid) component, a watery (aqueous) component and a mucin (mucous-like) component. The oily (lipid) component, known as meibum, is produced by meibomian glands located in the eyelids. The watery component is produced lacrimal glands located behind the upper eyelids. The mucin component is produced by goblet cells located in the conjunctiva of the eye. Insufficiency or excess of any of these tear components can result in dry eye. For example, in patients who suffer from meibomian gland disfunction, insufficient meibum is produced which allows the tear film to evaporate too rapidly, thereby resulting in an evaporative dry eye condition. If the lacrimal glands fail to produce enough of the watery component, the overall volume or tears may be reduced and the eyes will not be kept sufficiently moist, thereby resulting in aqueous deficiency type of dry eye. Also, other underlying conditions and phenomena, such as allergic or inflammatory disorders, hormonal changes, various behavioral and environmental factors, diabetes, prolonged contact lens wear, advanced age, certain autoimmune diseases (e.g. Sjogren's Syndrome and Systemic Lupus Erythematosus), ocular surgeries including PRK or LASIK, medications, computer use, ocular fatigue, corneal sensitivity, pterygium and eyelid irregularities (e.g. ptosis, entropion/ectropion, pinguecula), may also cause or contribute to Dry Eye.

Dry Eye is a chronic Dry Eye Disease (DED) is a chronic inflammatory disease of the lacrimal gland and ocular surface tissue (epithelial cell, goblet cells, Meibomian gland, etc.). Stress to the ocular surface (environmental factors, systemic diseases, infection, endogenous stress, antigens, genetic factors) is postulated as the pathogenic triggering mechanism. Proinflammatory cytokines, chemokines infiltrate the ocular surface and lacrimal gland thereby resulting in a cycle of damage to the ocular surface and inflammation. Dry Eye is typically associated with inflammatory changes in parts of the eye such as the adnexa, conjunctiva and/or cornea. A number of potential

Dry Eye treatments designed to inhibit various inflammatory pathways have been studied. Drugs which have heretofore been approved for treatment of Dry Eye include Lifitegrast (Xiidra®, Shire US Inc., Lexington, Mass.) and Cyclosporine (Restasis® and Restasis MultiDose®, Allergan, Inc. Irvine, Calif.; Cega™; Sun Pharmaceutical industries, ltd., Princeton, N.J.).

Applicant is developing Risuteganib, a non-natural peptide having the molecular formula C22-H39-N9-O11-S and the following structural formula:

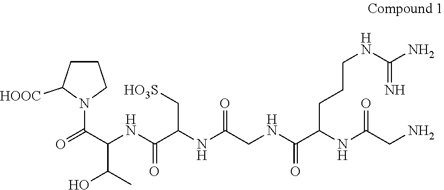 [SMILES: CC(O)C(NC(=O)C(CS(=O)(=O)O)NC(=O)CNC(=O)C(CCCNC(=N)N)NC(=O)CN)C(=O)N1CCCC1C(=O)O]

Risuteganib has also been referred to by other names, nomenclatures and designations, including: ALG-1001; Glycyl-L-arginylglycyl-3-sulfo-L-alanyl-L-threonyl-L-proline; Arg-Gly-NH—CH(CH₂—SO₃H)COOH; and Luminate® (Allegro Ophthalmics, LLC, San Juan Capistrano, Calif.).

Risuteganib is an anti-integrin which has been shown to target a number of integrins upstream in the oxidative stress pathway. Risuteganib acts broadly to downregulate multiple angiogenic and inflammatory processes, including those associated with hypoxia and oxidative stress. Risuteganib is presently known to cause a number of effects, including the following:
Deterrence of angiogenesis and possible regression of neovascularization by downregulating production of VEGF and other proangiogenic growth factors including ANG-2;
Reduction of vascular leakage by inhibiting the production of VEGF and inflammatory mediators;
Reduction of inflammation, at least in part by targeting multiple integrin subunits, including reducing expression of the Complement 3 Receptor (also known as Integrin αMβ2). This results in reduced leucocyte adhesion, reduced trans-endothelial leucocyte migration and resultant reduction in the symptoms of inflammation; and
Neuroprotection evidenced by decreased apoptosis, increased cell survival in the ROP Model, reduction of free radical oxygen production and enhanced mitochondrial health.
   Additional description of and information relating to Risuteganib is provided in U.S. Pat. Nos. 9,018,352; 9,872,886; 9,896,480; 10,307,460; 10,639,347; and 10,590,166 and in United States Patent Application Publication Nos. 2018/0207227 and 2019/0062371, the entire disclosure of each such patent and patent application being expressly incorporated herein by reference.

### SUMMARY

As used herein, the term “patient or “subject” refers to either a human or non-human animals, such as humans, primates, mammals, and vertebrates.

The term “treat” or “treating” refers to preventing, eliminating, curing, deterring, reducing the severity or reducing at least one symptom of a condition, disease or disorder.

The phrase “effective amount” or “amount effective to” refers to an amount of an agent that produces some desired effect at a reasonable benefit/risk ratio. In certain embodiments, the term refers to that amount necessary or sufficient to treat Dry Eye or a symptom of Dry Eye. The effective amount may vary depending on such factors as the disease or condition being treated, the particular composition being administered, or the severity of the disease or condition. One of skill in the art may empirically determine the effective amount of a particular agent without necessitating undue experimentation.

In accordance with the present disclosure, there are provided compositions and methods for treating Dry Eye in a human or non-human animal subject wherein an effective amount of a pharmaceutical composition comprising an anti-integrin peptide is administered to an eye of the subject. In some embodiments, the anti-integrin peptide may comprise a peptide which causes at least one effect selected from: reduced expression of the Complement 3 Receptor (Integrin αMβ2); reduced leucocyte adhesion; and reduced trans-endothelial leucocyte migration. In some embodiments, the peptide may comprises Risuteganib. In some embodiments the peptide may comprise a peptide other than Risuteganib which exhibits these specified effect(s), such as the active peptides disclosed in the above-incorporated United States Patent Application Publication No. 2019/0062371 entitled Peptide Compositions and Related Methods.

In accordance with another aspect of the present disclosure, the pharmaceutical composition may comprise a solution, suspension or gel suitable for topical administration to an eye which contain the anti-integrin peptide in any suitable carrier such as saline solution or artificial tears. The carrier may include one or more components known in the art of formulating compositions for topical administration to an eye, including but not necessarily limited to solvents, tonicitiy agents, buffering agents, preservative agents, surfactants, lubricants, excipients and pH adjusting agents.

In accordance with yet another aspect of the present disclosure, the pharmaceutical composition may, optionally, further comprise active (e.g., effective to treat Dry Eye) or inactive (e.g., effective as an excipient, lubricant or other formulation component) amount(s) of one or more of: a) amino acid(s) selected from: taurine, methionine and cysteine and/or b) a hyaluronan (e.g., hyaluronic acid, sodium hyaluronate, potassium hyaluronate, other salts of hyaluronic acid.). In some embodiments such amino acid and/or hyaluronan component(s) may be present in concentrations ranging from 0.125 to 5.0% by weight of the formulation.

In accordance with yet another aspect of the present disclosure, there are provided pharmaceutical compositions for topical administration to an eye comprising Risuteganib and taurine in amounts which render the pharmaceutical composition effective to treat Dry Eye.

In accordance with yet another aspect of the present disclosure, there are provided pharmaceutical compositions for topical administration to an eye comprising Risuteganib and a hyaluronan (e.g., hyaluronic acid, sodium hyaluronate, potassium hyaluronate, other salts of hyaluronic acid.) in amounts which render the pharmaceutical composition effective to treat Dry Eye.

In accordance with yet another aspect of the present disclosure, there are provided pharmaceutical compositions for topical administration to an eye comprising Risuteganib, a hyaluronan (e.g., hyaluronic acid, sodium hyaluronate, potassium hyaluronate, and other salts of hyaluronic acid.) and an amino acid selected from taurine, methionine and cysteine, in amounts which render the pharmaceutical composition effective to treat Dry Eye.

Still further aspects and details of the present invention will be understood upon reading of the detailed description and examples set forth herebelow

### BRIEF DESCRIPTION OF THE DRAWINGS

The following drawings are included in this patent application and referenced in the following Detailed Description. These drawings are intended only to illustrate certain aspects or embodiments of the present disclosure and do not limit the scope of the present disclosure in any way:

FIG. 1 is a graphic showing expression of integrin αMβ2 in ischemic retinopathy of prematurity (ROP) mice after Risuteganib treatment.

FIG. 2 is a copy of the SICCA Ocular Staining Score form as published by the Sjögrens International Collaboration Clinical Alliance (SICCA).

FIG. 3 shows a visual analog scale (VAS) symptom index used in the Human Study described below.

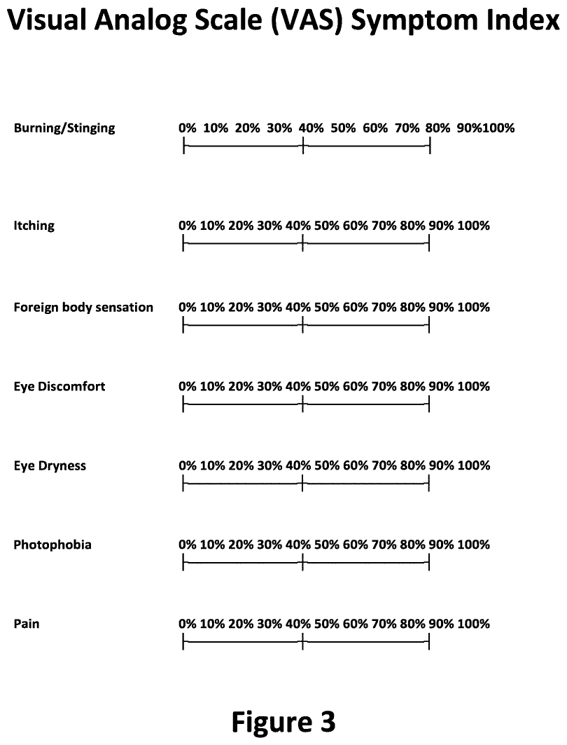

FIG. 4 is a graph of mean Tear Breakup Time (TBUT) (seconds) vs. time (weeks) in subjects treated with Test Formula 1 containing 0.6% Taurine and 0.6% Risuteganib 0.6% in the Human Study described below compared to historical control values.

FIG. 5 is a graph of mean Total Ocular Staining Score vs. time (weeks) in subjects treated with Test Formula 1 containing 0.6% Taurine and 0.6% Risuteganib 0.6% in the Human Study described below compared to historical control values.

FIG. 6 is a graph of mean Corneal Staining Score vs. time (weeks) in subjects treated with Test Formula 1 containing 0.6% Taurine and 0.6% Risuteganib 0.6% in the Human Study described below compared to historical control values.

FIG. 7 is a graph of mean Nasal Conjunctival Staining Score vs. time (weeks) in subjects treated with Test Formula 1 containing 0.6% Taurine and 0.6% Risuteganib 0.6% in the Human Study described below compared to historical control values.

FIG. 8 is a graph of mean composite VAS score (all symptoms) in subjects treated with Test Formula 1 containing 0.6% Taurine and 0.6% Risuteganib 0.6% in Human Study A (described below) compared to historical control values.

### DETAILED DESCRIPTION

The following detailed description and the accompanying drawings to which it refers are intended to describe some, but not necessarily all, examples or embodiments of the present disclosure. The described embodiments are to be considered only as illustrative and not restrictive. The contents of this detailed description and the accompanying drawings do not limit the scope of the invention in any way.

As used in this patent application, the term ALG-1007 is used to refer generally to pharmaceutical preparations that contain Risutiganib as an active agent for topical administration to the eye to treat eye disorders including Dry Eye. Non-limiting examples of possible ALG-1007 formulations include the following:

Example 1

| | |
|---|---|
| Purified Water | q.s. to 100% |
| Sodium Hyaluronate | 0.125% |
| Carboxymethylcellulose | 0.2% |
| Sodium Alginate | 0.05% |
| Sodium Chloride | 0.20% |
| Potassium Chloride | 0.14% |
| Magnesium Chloride | 0.011% |
| Boric Acid | 0.2% |
| Sodium Chlorite | 0.05% |
| Hydrogen Peroxide | 0.017% |
| Taurine | 0.6-5.0% |
| Risuteganib | 0.6-5.0% |
| 1N HCL or 1N NaOH | as needed to pH 7.0-7.4 |

Example 2

| | |
|---|---|
| Purified Water | q.s. to 100% |
| Sodium Hyaluronate | 0.125% |
| Carboxymethylcellulose | 0.2% |
| Sodium Alginate | 0.05% |
| Sodium Chloride | 0.20% |
| Potassium Chloride | 0.14% |
| Magnesium Chloride | 0.011% |
| Potassium Phosphate | 0.1% |
| (Monobasic, Unhydrous) | |
| Potassium Phosphate | 0.4% |
| (Dibasic, Unhydrous) | |
| Sodium Chlorite | 0.05% |
| Hydrogen Peroxide | 0.017% |
| Taurine | 0.6-5.0% |
| Risuteganib | 0.6-5.0% |
| 1N HCL or 1N NaOH | as needed to pH 7.0-7.4 |

Example 3:

| | |
|---|---|
| Purified Water | q.s. to 100% |
| Sodium Hyaluronate | 0.125% |
| Carboxymethylcellulose | 0.01%-10% |
| Sodium Alginate | 0.01%-15% |
| Sodium Chloride | 0.20% |
| Potassium Chloride | 0.14% |
| Magnesium Chloride | 0.011% |
| Boric Acid | 0.2% |
| Sodium Chlorite | 0.05% |
| Hydrogen Peroxide | 0.017% |
| Taurine | 0.6-0.8% |
| Risuteganib | 0.6-0.8% |
| 1N HCL or 1N NaOH | as need to pH 7.0-7.4 |

Example 4:

| | |
|---|---|
| Purified Water | q.s. to 100% |
| Carboxymethylcellulose | 0.2% |
| Sodium Alginate | 0.05% |
| Sodium Chloride | 0.20% |
| Potassium Chloride | 0.14% |
| Magnesium Chloride | 0.011% |
| Boric Acid | 0.2% |
| Sodium Chlorite | 0.05% |
| Hydrogen Peroxide | 0.017% |
| Taurine | 0.6-0.8% |
| Risuteganib | 0.6-0.8% |
| 1N HCL or 1N NaOH | as need to pH 7.0-7.4 |

Example 5:

| | |
|---|---|
| Purified Water | q.s. to 100% |
| Carboxymethylcellulose | 0.01%-10% |
| Sodium Alginate | 0.01%-15% |
| Sodium Chloride | 0.20% |
| Potassium Chloride | 0.14% |
| Magnesium Chloride | 0.011% |
| Boric Acid | 0.2% |
| Sodium Chlorite | 0.05% |
| Hydrogen Peroxide | 0.017% |
| Taurine | 0.6-0.8% |
| Risuteganib | 0.6-0.8% |
| 1N HCL or 1N NaOH | as need to pH 7.0-7.4 |

Example 6:

| | |
|---|---|
| Purified Water | q.s. to 100% |
| Sodium Hyaluronate | 0.125% |
| Carboxymethylcellulose | 0.2% |
| Sodium Alginate | 0.05% |
| Sodium Chloride | 0.20% |
| Potassium Chloride | 0.14% |
| Magnesium Chloride | 0.011% |
| Boric Acid | 0.2% |
| Sodium Chlorite | 0.05% |
| Hydrogen Peroxide | 0.017% |
| Risuteganib | 0.6-0.8% |
| 1N HCL or 1N NaOH | as need to pH 7.0-7.4 |

Example 7:

| | |
|---|---|
| Purified Water | up to 100% |
| Sodium Hyaluronate | 0.125% |
| Carboxymethylcellulose | 0.01%-10% |
| Sodium Alginate | 0.01%-15% |
| Sodium Chloride | 0.20% |
| Potassium Chloride | 0.14% |
| Magnesium Chloride | 0.011% |
| Boric Acid | 0.2% |
| Sodium Chlorite | 0.05% |
| Hydrogen Peroxide | 0.017% |
| Risuteganib | 0.6-0.8% |
| 1N HCL or 1N NaOH | as need to pH 7.0-7.4 |

Example 8:

| | |
|---|---|
| Purified Water | up to 100% |
| Sodium Alginate | 0.075% |
| Sodium Chloride | 0.20% |
| Potassium Chloride | 0.14% |
| Magnesium Chloride | 0.011% |
| Boric Acid | 0.2% |
| Sodium Chlorite | 0.05% |
| Hydrogen Peroxide | 0.017% |
| Risuteganib | 0.6-0.8% |
| 1N HCL or 1N NaOH | as need to pH 7.0-7.4 |

Example 9:

| | |
|---|---|
| Purified Water | up to 100% |
| Sodium Alginate | 0.01%-15% |
| Sodium Chloride | 0.20% |
| Potassium Chloride | 0.14% |
| Magnesium Chloride | 0.011% |
| Boric Acid | 0.2% |
| Sodium Chlorite | 0.05% |
| Hydrogen Peroxide | 0.017% |
| Risuteganib | 0.6-0.8% |
| 1N HCL or 1N NaOH | as need to pH 7.0-7.4 |

Example 10:

| | |
|---|---|
| Purified Water | q.s. to 100% |
| Sodium Alginate | 0.01%-15% |
| Sodium Chloride | 0.20% |
| Potassium Chloride | 0.14% |
| Magnesium Chloride | 0.011% |
| Boric Acid | 0.2% |
| Sodium Chlorite | 0.05% |
| Hydrogen Peroxide | 0.017% |
| Taurine | 0.6-0.8% |
| Risuteganib | 0.6-0.8% |
| 1N HCL or 1N NaOH | as need to pH 7.0-7.4. |

Example 11:

| | |
|---|---|
| Purified Water | q.s. to 100% |
| Sodium Alginate | 0.01%-15% |
| Sodium Chloride | 0.20% |
| Potassium Chloride | 0.14% |
| Magnesium Chloride | 0.011% |
| Boric Acid | 0.2% |
| Sodium Chlorite | 0.05% |
| Hydrogen Peroxide | 0.017% |
| Risuteganib | 0.6-0.8% |
| 1N HCL or 1N NaOH | as need to pH 7.0-7.4. |

Example 12: (Referred to below as ALG-1007 Test Formula 1)

| | |
|---|---|
| Purified Water | q.s. to 100% |
| Sodium Hyaluronate | 0.125% |
| Carboxymethylcellulose | 0.2% |
| Sodium Alginate | 0.05% |
| Sodium Chloride | 0.20% |
| Potassium Chloride | 0.14% |
| Magnesium Chloride | 0.011% |
| Boric Acid | 0.2% |
| Sodium Chlorite | 0.05% |
| Hydrogen Peroxide | 0.017% |
| Taurine | 0.125-0.6% |
| Risuteganib | 0.125-0.6% |
| 1N HCL or 1N NaOH | as needed to pH 7.0-7.4 |

The Taurine component of each of the disclosed formulations is optional. Others have noted that taurine may, itself, have some efficacy in treating Dry Eye as described in United States Patent Application Publication No. 2008/0261890 (Ousler et al.) Use of Neurotransmitters and Neuropeptides for the Treatment of Dry Eye and Related Conditions. Should it be determined that taurine is to any extent, an active component of a particular formulation that contains the optional taurine, the relative amounts of Risuteganib and taurine may vary and may be optimized for treatment of Dry Eye. The examples shown above are merely examples and are not intended to exhaustively describe all possible formulations that may be used in accordance with this disclosure. In alternative versions of the above formulation Examples 1-12 or any other embodiments in which taurine (or alternatively methionine or cysteine) is present, such component may be present in an amount that ranges from 0.125 to 5.0% by weight of the formulation.

In embodiments which do include the optional taurine, the taurine may be fully or partially replaced with methionine or cysteine. The present disclosure includes the above-shown, non-limiting Examples 1 through 12 wherein the taurine is replaced by methionine or cysteine at the concentration levels indicated or in other amounts determined to be effective or suitable.

In some embodiments taurine component (e.g., taurine or, alternatively, methionine or cysteine) may be absent and in other embodiments the relative amounts of Risuteganib and the taurine component Taurine may vary. In some formulation embodiments wherein both Risuteganib and a taurine component are present, the Risuteganib may be present in the range of 0.05% to 5.0% and the taurine component may be present in the range of 0.05% to 5.0%. The following are non-limiting examples of low, medium and high concentration formulations:
Low Formulation: 0.05% of Risuteganib+0.05% taurine component.
Mid Formulation: 0.6%-0.8% Risuteganib+0.6-0.8% taurine component.
High Formulation: 5% Risuteganib+5% taurine component.

In embodiments in which both Risuteganib and a taurine component are present, the Risuteganib and the taurine component may be combined in a single solution as in formulation Examples 1 through 9 above. Alternatively, the Risuteganib and the taurine component may be provided separate solutions or compositions and may be administered concurrently or at differing times.

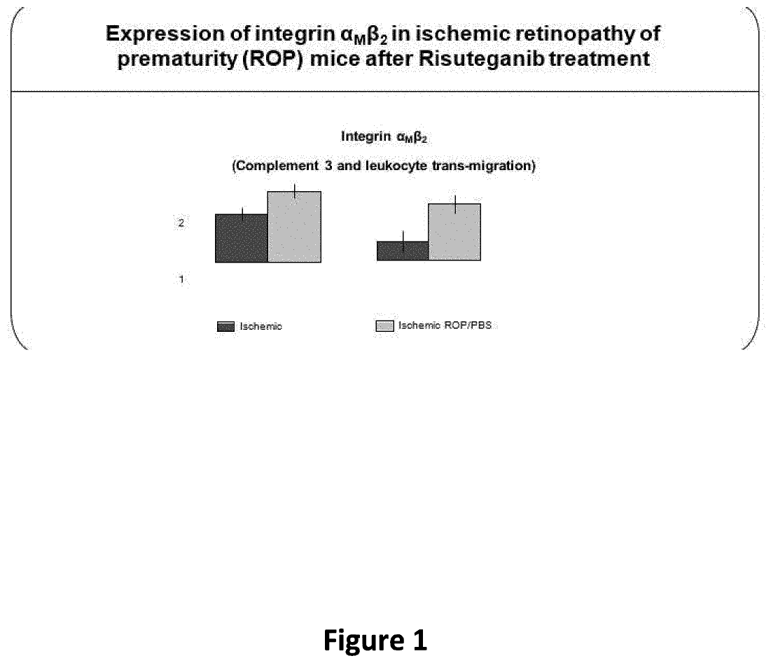

Risuteganib is a small peptide that acts as an integrin inhibitor. Risuteganib targets multiple integrin subunits, including Integrin αMβ2 which is sometimes referred to as the “compliment 3 receptor” and is actively involved in inflammatory pathways and, in particular, the compliment 3 pathway. As shown in FIG. 1, Risuteganib decreases expression of Integrin αMβ2. By causing such inhibition of Integrin αMβ2 Risuteganib may interfere with leucocyte adhesion and trans-endothelial migration, thereby resulting in decreased inflammation and improvement in the symptoms of Dry Eye.

In some embodiments, the sodium hyaluronate or other hyaluronan component may be reduced or entirely eliminated from the formulation. In alternative versions of the above formulation Examples 1-12 or in any other embodiments in which sodium hyaluronate or another hyaluronan component (e.g., potassium hyaluronate, hyaluronic acid) is present, such component may be present in an amount ranging from 0.125 to 5.0% by weight of the formulation.

Also, in alternative versions of any of the above formulation Examples 1-12, or in any other embodiment, the pH of the formulation or pharmaceutical composition may be in a range from 6.5 to 8.0.

Risuteganib also downregulates oxidative stress response.

### Mouse Study

Female C57BL/6 mice were randomly divided into four (4) groups and treated as shown in TABLE 1, below:

TABLE 1

| Group | Treatment |
|---|---|
| 1 | Control: Desiccating Stress/No Scopolamine |
| 2 | Scopolamine Control: Desiccating Stress + Scopolamine |
| | 10 mg/Kg, i.p. (in 0.2 ml injectate volume) administered |
| | once per day for 10 days |
| 3 | Scopolamine/Saline Control: Desiccating Stress + |
| | Scopolamine 10 mg/Kg, i.p. (in 0.2 ml injectate volume) |
| | administered once per day for 10 days + Vehicle* (control) |
| | 0.2 ml, i.p. administered once per day for 10 days |
| 4 | Scopolamine/Saline + ALG-1007 Test Formula 1: |
| | Desiccating Stress + Scopolamine 10 mg/Kg, i.p. (in 0.2 ml |
| | injectate volume) administered once per day for 10 days + 0.6 |
| | mg (1 drop) ALG-1007 Test Formula 1 (Example 12 above) |
| | containing 0.6% Taurine and 0.6% Risuteganib; administered |
| | topically to the eyes 2 times per day for 10 days |

*Vehicle (control) contained all components of the Test Formulation (Example 5 above) except for Risuteganib and Taurine.

Dry eye was induced by exposing the mice to desiccating stress conditions using a controlled environmental chamber for twelve (12) consecutive days. After twelve (12) days, the animals were removed from the desiccating conditions, the eyes were stained with fluoresein and photographed. Visual assessment of the photographs revealed intense Fluoresein staining of the cornea in control (Groups 1, 2 and 3) eyes, while the eyes of animals treated with ALG-1007 Test Formula 1 (Group 4) did not exhibit fluorescein staining of the cornea, thereby indicating healthy corneas in Group 4.

After sacrifice of the animals, corneal slices were prepared and processed for immunochemistry to measure corneal concentration of the following: Interleukin 1β, Interleukin 6, TNF-α, Glial acidic fibrillary protein (GFAP), 18 kDa Translocator Protein (TSPO), Caspase 6, Caspace 9, Superoxide dismutase (SOD), Glutathione peroxidase and Catalase. The results of these analyses are summarized in Table 2, below:

TABLE 2

| Marker | | Corneal Levels |
|---|---|---|
| Interleukin-1β (IL-1β) | immune response, | Reduced |
| | inflammation | |
| Interleukin-6 (IL-6) | immune response, | Reduced |
| | inflammation | |
| TNF-α | inflammation | Reduced |
| GFAP—Glial acidic | neurodegeneration | Reduced |
| fibrillary protein | | |
| TSPO—18 kDa | apoptosis | Reduced |
| Translocator Protein | | |
| Caspase 6 | apoptosis | Reduced |
| Caspase 9 | apoptosis | Reduced |
| SOD—Superoxide | antioxidant | Increased |
| dismutase* | | |
| Glutathione peroxidase* | antioxidant | Increased |
| Catalase* | antioxidant | Increased |

The animals in Treatment Group 4 had lower corneal concentrations of Interleukin 1β, Interleukin 6, TNF-α, Glial acidic fibrillary protein (GFAP), Caspase 6 and Caspace 9 as well as higher Superoxide dismutase (SOD), Glutathione peroxidase and Catalase than the animals in Groups 1, 2 or 3. This indicates that ALG-1007 Test Formula 1 caused an anti-inflammatory effect in this study.

### Human Stud

An initial human study was conducted to explore the safety and efficacy of ALG-1007 Test Formula 1 applied topically to the eyes of subjects suffering from Dry Eye. In selecting subjects for this study, the following exclusion criteria were applied:
Known allergies to any of the drug ingredient;
Wears contact lenses or unwilling to stop wearing contact lenses;
Pregnant, nursing or lactating;
Current ocular infection, inflammation or acute allergic conjunctivitis;
History of: ocular herpetic keratitis, ocular surgery in the past 6 months, LASIK surgery, use of glaucoma medicine;
Subjects with DED secondary to scarring or destruction of conjunctival goblet cells (i.e. chemical burn);
Eyelid abnormalities or extensive ocular scarring;
Use of any topical medication or antibiotic for the;
treatment of blepharitis or meibomian gland disease;
Current use of active DED treatment (i.e. lifitegrast, cyclosporine, mast cell stabilizers, anti-histamine, corticosteroids) (Use of artificial tears or eye lubricants was allowed);
Participation in any investigational drug (within 60 days) or device study (within 30 days) prior to baseline.

Subjects were randomly assigned to treatment groups, as follows:

| Treatment | |
|---|---|
| Group | Treatment |
| 1 | Dosage Level 1: Test Formula 1 (Example 5 above) |
| | containing 0.125% Taurine and 0.125% Risuteganib. |
| 2 | Dosage Level 2: Test Formula 1 (Example 5 above) |
| | containing 0.25% Taurine and 0.25% Risuteganib. |
| 3 | Dosage Level 3: Test Formula 1 (Example 5 above) |
| | containing 0.4% Taurine and 0.4% Risuteganib. |
| 4 | Dosage Level 4: Test Formula 1 (Example 5 above) |
| | containing 0.6% Taurine and 0.6% Risuteganib. |

One (1) drop of test solution was administered topically to each eye of each subject twice per day (morning and evening) for 12 weeks. Tear break-up time (TBUT), conjunctival staining and corneal staining and Dry Eye symptoms were measured at the following time points: zero time, week 1, week 2, week 4, week 6, week 8, week 10 and week 12.

### Assessment of Tear Breakup Time:

Tear breakup time (TBUT) is determined by measuring the interval between instillation of topical fluorescein 0.5% and appearance of the first dry spots on the cornea. Measure it prior to instillation of any anesthetic eye drops. A fluorescein strip is moistened with saline and applied to the inferior cul-de-sac. After several blinks, the tear film is observed for appearance of the first dry spots on the cornea.

The following Table 3 shows the mean change in TUBT (seconds)+/−Standard Deviation of the Mean for each treatment group at each time point from Week 1 through Week 12:

TABLE 3

| | TBUT | TBUT | TBUT | TBUT | TBUT | TBUT | TBUT | TBUT |
|---|---|---|---|---|---|---|---|---|
| | Week 0 | Week 1 | Week 2 | Week 4 | Week 6 | Week 8 | Week 10 | Week 12 |
| Treatment | (Mean ± | (Mean ± | (Mean ± | (Mean ± | (Mean ± | (Mean ± | (Mean ± | (Mean ± |
| Group | SD) | SD) | SD) | SD) | SD) | SD) | SD) | SD) |
| 1 | 0.00 ± | 0.50 ± | 0.50 ± | 1.10 ± | 1.77 ± | 2.00 ± | 2.87 ± | 1.97 ± |
| | 0.0 | 0.26 | 0.25 | 0.63 | 0.57 | 0.52 | 0.44 | 0.48 |
| 2 | 0.00 ± | 0.07 ± | 0.17 ± | 1.23 ± | 1.33 ± | 2.30 ± | 3.30 ± | 3.37 ± |
| | 0.0 | 0.16 | 0.14 | 0.52 | 0.53 | 0.97 | 0.80 | 0.85 |
| 3 | 0.00 ± | 0.57 ± | −0.47 ± | 0.43 ± | 1.10 ± | 1.93 ± | 0.67 ± | 0.50 ± |
| | 0.0 | 0.12 | 0.21 | 0.48 | 0.33 | 1.17 | 0.40 | 0.48 |
| 4 | 0.00 ± | 0.10 ± | 1.97 ± | 2.70 ± | 4.77 ± | 5.27 ± | 6.43 ± | 6.67 ± |
| | 0.0 | 0/05 | 0.34 | 0.28 | 0.29 | 0.25 | 0.70 | 0.67 |

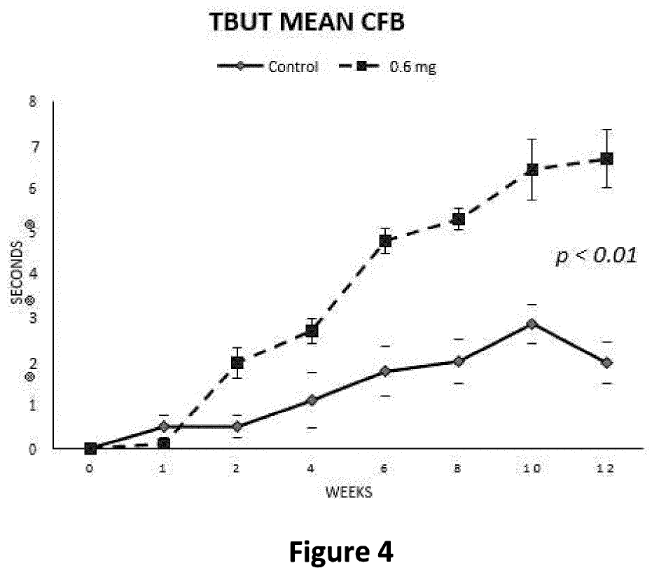

Mean TBUT scores for each treatment group were compared to historical control data. While some effect on TBUT was apparent at each of the dosage levels tested, the most significant was seen in the Treatment Group 4 subjects who were treated with the 0.6% Taurine/0.6% Risuteganib dosage level. FIG. 4 is a graph showing mean TBUT for Treatment Group 4 subjects in comparison to historical control data.

### Assessment of Ocular Staining:

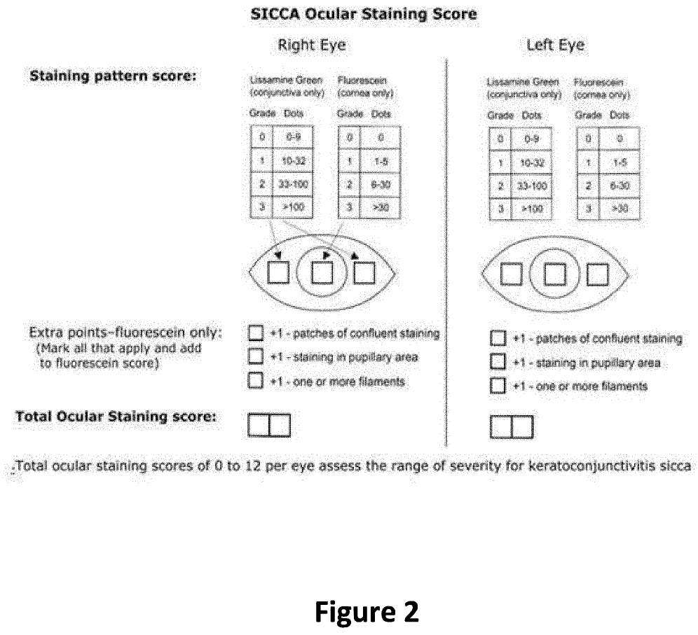

Total Ocular Staining Scores (TOSS) were calculated using the SICCA Ocular Staining Score system shown in FIG. 2. The following Table 4 shows the mean change in TOSS for each treatment group at each time point from Week 1 though Week 12:

TABLE 4

| | TOSS | TOSS | TOSS | TOSS | TOSS | TOSS | TOSS | TOSS |
|---|---|---|---|---|---|---|---|---|
| | Week 0 | Week 1 | Week 2 | Week 4 | Week 6 | Week 8 | Week 10 | Week 12 |
| Treatment | (Mean ± | (Mean ± | (Mean ± | (Mean ± | (Mean ± | (Mean ± | (Mean ± | (Mean ± |
| Group | SD) | SD) | SD) | SD) | SD) | SD) | SD) | SD) |
| 1 | 0.00 ± | −0.03 ± | −0.03 ± | −1.00 ± | 0.00 ± | 0.00 ± | 0.00 ± | 0.00 ± |
| | 0.0 | 3.00 | 0.00 | 5.00 | 0.00 | 0.00 | 0.00 | 0.00 |
| 2 | 0.00 ± | −0.6 ± | −0.6 ± | −1.90 ± | 0.00 ± | 0.00 ± | 0.00 ± | 0.00 ± |
| | 0.0 | 10.0 | 0.0 | 10.0 | 0.0 | 0.0 | 0.0 | 0.0 |
| 3 | 0.00 ± | 75.00 ± | 75.00 ± | 59.00 ± | 0.00 ± | 0.00 ± | 0.00 ± | 0.00 ± |
| | 0.0 | 90.0 | 90.0 | 90.0 | 0.0 | 0.0 | 0.0 | 0.0 |
| 4 | 0.00 ± | 58.00 ± | 95.00 ± | 65.00 ± | 0.00 ± | 0.00 ± | 0.00 ± | 0.00 ± |
| | 0.0 | 0.0 | 70.0 | 60.0 | 0.0 | 0.0 | 0.0 | 0.0 |

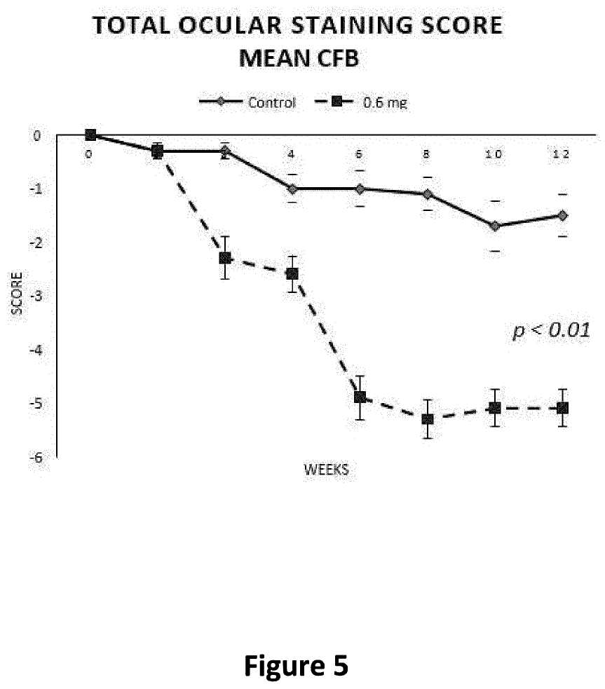

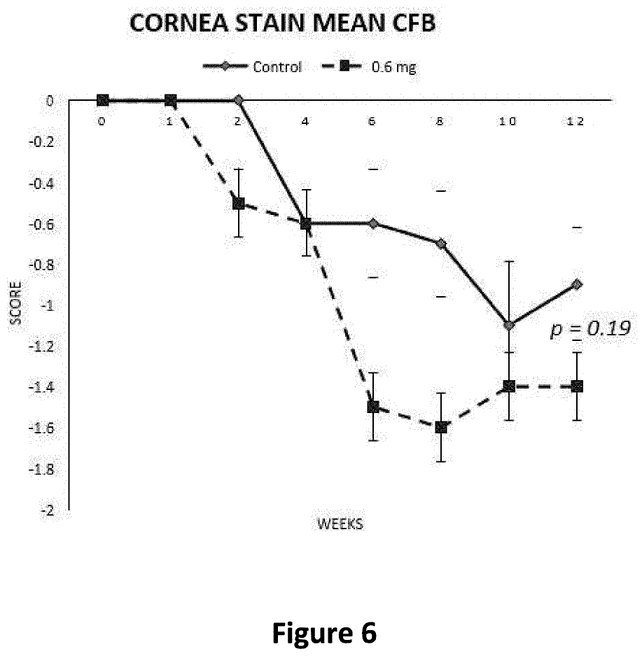

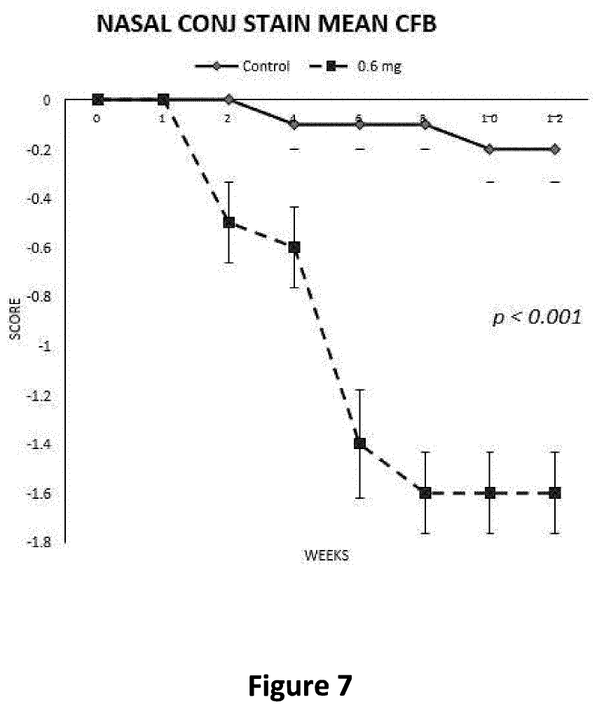

Mean TOSS scores for each treatment group were compared to historical control data. Effects on TOSS were apparent at the 1 week, 2 week and 4 week time points in subjects of Treatment Groups 3 (0.4% Taurine/0.4% Risuteganib) and 4 (0.6% Taurine/0.6% Risuteganib). The graph of FIG. 5 shows mean TOSS for Treatment Group 4 subjects at each time point in comparison to historical control data. The mean corneal staining score for Treatment Group 4 subjects at each time point are shown, in comparison to historical control data, in FIG. 6 and the mean nasal conjunctival staining score for Treatment Group 4 subjects at each time point are shown, in comparison to historical control data, in FIG. 7. These data indicate that at least the TOSS and Nasal Conjunctival staining scores were significantly effected in at least the Treatment Group 4 subjects.

### Assessment of Dry Eye Symptoms:

The following Dry Eye symptoms were assessed in each subject at each time point using a Visual Analog Scale (VAS):
Burning/Stinging
Itching
Foreign Body Sensation
Eye Discomfort
Eye Dryness
Photophobia
Pain

The following Table 5 shows mean change in VAS score for the “Eye Dryness” symptom for each treatment group at each time point from Week 1 through Week 12:

TABLE 4

| | TOSS | TOSS | TOSS | TOSS | TOSS | TOSS | TOSS | TOSS |
|---|---|---|---|---|---|---|---|---|
| | Week 0 | Week 1 | Week 2 | Week 4 | Week 6 | Week 8 | Week 10 | Week 12 |
| Treatment | (Mean ± | (Mean ± | (Mean ± | (Mean ± | (Mean ± | (Mean ± | (Mean ± | (Mean ± |
| Group | SD) | SD) | SD) | SD) | SD) | SD) | SD) | SD) |
| 1 | 0.00 ± | −3.50 ± | −3.60 ± | −14.10 ± | −16.00 ± | −23.80 ± | −40.20 ± | −40.10 ± |
| | 0.0 | 2.36 | 2.35 | 3.59 | 4.94 | 6.73 | 9.06 | 8.86 |
| 2 | 0.00 ± | −1.00 ± | −6.00 ± | −8.90 ± | −6.00 ± | −5.50 ± | −22.70 ± | −28.30 ± |
| | 0.0 | 0.42 | 4.92 | 8.63 | 9.06 | 8.72 | 11.0 | 6.26 |
| 3 | 0.00 ± | −10.00 ± | −22.00 ± | −46.00 ± | −52.00 ± | −58.00 ± | −62.00 ± | −68.40 ± |
| | 0.0 | 0.0 | 5.73 | 9.33 | 8.27 | 6.11 | 5.73 | 5.76 |
| 4 | 0.00 ± | −2.90 ± | −12.10 ± | −13.30 ± | −50.50 ± | −55.60 ± | −54.60 ± | −55.10 ± |
| | 0.0 | 0.90 | 2.13 | 2.06 | 2.97 | 2.27 | 2.11 | 2.34 |

The assessed symptom of Eye Dryness was reduced in all Treatment Groups 1, 2 3 and 4.

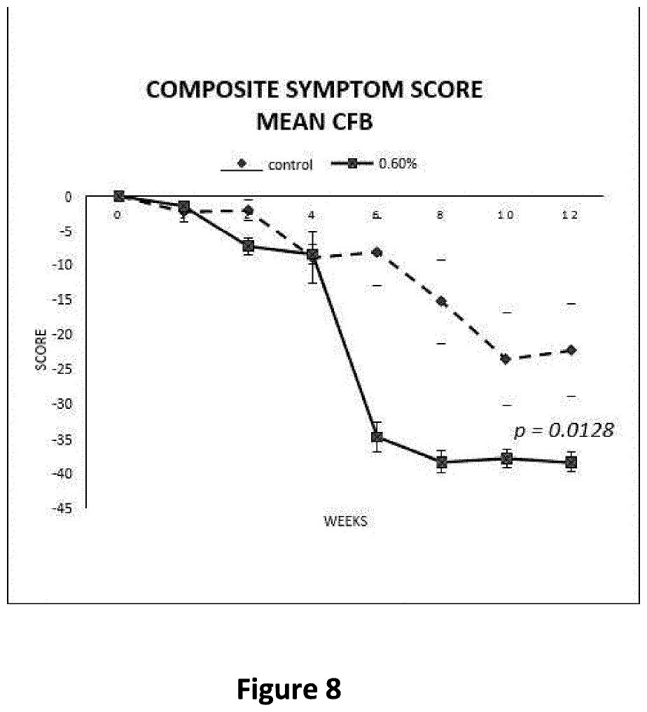

FIG. 8 is a graph showing mean change in composite VAS scores (all assessed symptoms) in Treatment Group 4 subjects (0.6% Taurine+0.6% Risuteganib) compared to historical control values.

No significant toxicity or untoward effects were observed in the subjects treated in this human study.

The detailed description set forth above makes reference to certain examples or embodiments of the invention. However, various additions, deletions, alterations and modifications may be made to those examples and embodiments without departing from the intended spirit and scope of the invention. For example, any element or attribute of one embodiment or example may be incorporated into or used with another embodiment or example, unless otherwise specified of if to do so would render the embodiment or example unsuitable for its intended use. Also, where the steps of a method or process have been described or listed in a particular order, the order of such steps may be changed unless otherwise specified or unless doing so would render the method or process unworkable for its intended purpose. All reasonable additions, deletions, modifications and alterations are to be considered equivalents of the described examples and embodiments and are to be included within the scope of the following claims.

1. A method for treating Dry Eye in a subject in need thereof, said method comprising the step of administering to the subject an effective amount of a pharmaceutical composition comprising an anti-integrin peptide.

2. A method according to claim 1 wherein the peptide causes at least one effect selected from:
reduced expression of the Complement 3 Receptor (Integrin αMβ2);
reduced leucocyte adhesion; and
reduced trans-endothelial leucocyte migration.

3. A method according to claim 1 wherein the peptide comprises Risuteganib.

4. A method according to 3 wherein the pharmaceutical composition comprises 0.05 to 5.0% by weight Risuteganib.

5. A method according to claim 1 wherein the method further comprises administering to the eye an effective amount of at least one amino acid selected from: taurine, methionine and cysteine.

6. A method according to claim 5 wherein said at least one amino acid is/are included in the pharmaceutical composition.

7. A method according to claim 1 wherein the pharmaceutical composition further comprises an effective amount of taurine.

8. A method according to claim 1 further comprising administering to the eye an effective amount of at least one hyaluronan.

9. A method according to claim 8 wherein said at least one hyaluronan is/are included in the pharmaceutical composition.

10. A method according to claim 1 wherein the pharmaceutical composition comprises Risuteganib, taurine and sodium hyaluronate, in a carrier suitable for said topical administration to the eye.

11. A method according to claim 1 wherein the pharmaceutical composition contains from 0.125 to 5.0% (by weight) of the anti-integrin peptide and from 0.125 to 5.0% taurine.

12. A method according to claim 1 wherein the pharmaceutical composition comprises:

| Purified Water | q.s. to 100% |
|---|---|
| Sodium Hyaluronate | 0.125% |
| Carboxymethylcellulose | 0.2% |
| Sodium Alginate | 0.05% |
| Sodium Chloride | 0.20% |
| Potassium Chloride | 0.14% |
| Magnesium Chloride | 0.011% |
| Boric Acid | 0.2%. |
| Sodium Chlorite | 0.05% |
| Hydrogen Peroxide | 0.017% |
| Taurine | 0.6-5.0% |
| Risuteganib | 0.6-5.0%. |

13. A method according to claim 1 wherein the pharmaceutical composition comprises:

| Purified Water | q.s. to 100% |
|---|---|
| Sodium Hyaluronate | 0.125% |
| Carboxymethylcellulose | 0.2% |
| Sodium Alginate | 0.05% |
| Sodium Chloride | 0.20% |
| Potassium Chloride | 0.14% |
| Magnesium Chloride | 0.011% |
| Potassium Phosphate | 0.1% |
| Monobasic, Unhydrous Potassium Phosphate | 0.4% |
| Dibasic, Unhydrous Sodium Chlorite | 0.05% |
| Hydrogen Peroxide | 0.017% |
| Taurine | 0.6-5.0% |
| Risuteganib | 0.6-5.0%. |

14. A method according to claim 1 wherein the pharmaceutical composition comprises:

| Purified Water | q.s. to 100% |
|---|---|
| Sodium Hyaluronate | 0.125% |
| Carboxymethylcellulose | 0.2% |
| Sodium Alginate | 0.05% |
| Sodium Chloride | 0.20% |
| Potassium Chloride | 0.14% |
| Magnesium Chloride | 0.011% |
| Boric Acid | 0.2% |
| Sodium Chlorite | 0.05% |
| Hydrogen Peroxide | 0.017% |
| Taurine | 0.6-0.8% |
| Risuteganib | 0.6-0.8%. |

15. A method according to claim 1 wherein the pharmaceutical composition comprises:

| Purified Water | q.s. to 100% |
|---|---|
| Sodium Hyaluronate | 0.125% |
| Carboxymethylcellulose | 0.01%-10% |
| Sodium Alginate | 0.01%-15% |
| Sodium Chloride | 0.20% |
| Potassium Chloride | 0.14% |
| Magnesium Chloride | 0.011% |
| Boric Acid | 0.2% |
| Sodium Chlorite | 0.05% |
| Hydrogen Peroxide | 0.017% |
| Taurine | 0.6-0.8% |
| Risuteganib | 0.6-0.8%. |

16. A method according to claim 1 wherein the pharmaceutical composition comprises:

| Purified Water | q.s. to 100% |
|---|---|
| Carboxymethylcellulose | 0.2% |
| Sodium Alginate | 0.05% |
| Sodium Chloride | 0.20% |
| Potassium Chloride | 0.14% |
| Magnesium Chloride | 0.011% |
| Boric Acid | 0.2% |
| Sodium Chlorite | 0.05% |
| Hydrogen Peroxide | 0.017% |
| Taurine | 0.6-0.8% |
| Risuteganib | 0.6-0.8%. |

17. A method according to claim 1 wherein the pharmaceutical composition comprises:

| Purified Water | q.s. to 100% |
|---|---|
| Carboxymethylcellulose | 0.01%-10% |
| Sodium Alginate | 0.01%-15% |
| Sodium Chloride | 0.20% |
| Potassium Chloride | 0.14% |
| Magnesium Chloride | 0.011% |
| Boric Acid | 0.2% |
| Sodium Chlorite | 0.05% |
| Hydrogen Peroxide | 0.017% |
| Taurine | 0.6-0.8% |
| Risuteganib | 0.6-0.8%. |

18. A method according to claim 1 wherein the pharmaceutical composition comprises:

| Purified Water | q.s. to 100% |
|---|---|
| Sodium Hyaluronate | 0.125% |
| Carboxymethylcellulose | 0.2% |
| Sodium Alginate | 0.05% |
| Sodium Chloride | 0.20% |
| Potassium Chloride | 0.14% |
| Magnesium Chloride | 0.011% |
| Boric Acid | 0.2% |
| Sodium Chlorite | 0.05% |
| Hydrogen Peroxide | 0.017% |
| Risuteganib | 0.6-0.8%. |

19. A method according to claim 1 wherein the pharmaceutical composition comprises:

| Purified Water | q.s. to 100% |
|---|---|
| Sodium Hyaluronate | 0.125% |
| Carboxymethylcellulose | 0.01%-10% |
| Sodium Alginate | 0.01%-15% |
| Sodium Chloride | 0.20% |
| Potassium Chloride | 0.14% |
| Magnesium Chloride | 0.011% |
| Boric Acid | 0.2% |
| Sodium Chlorite | 0.05% |
| Hydrogen Peroxide | 0.017% |
| Risuteganib | 0.6-0.8%. |

20. A method according to claim 1 wherein the pharmaceutical composition comprises:

| Purified Water | qs to 100% |
|---|---|
| Sodium Alginate | 0.075% |
| Sodium Chloride | 0.20% |
| Potassium Chloride | 0.14% |
| Magnesium Chloride | 0.011% |
| Boric Acid | 0.2% |
| Sodium Chlorite | 0.05% |
| Hydrogen Peroxide | 0.017% |
| Risuteganib | 0.6-0.8%. |

21. A method according to claim 1 wherein the pharmaceutical composition comprises:

| Purified Water | q.s. to 100% |
|---|---|
| Sodium Alginate | 0.01%-15% |
| Sodium Chloride | 0.20% |
| Potassium Chloride | 0.14% |
| Magnesium Chloride | 0.011% |
| Boric Acid | 0.2% |
| Sodium Chlorite | 0.05% |
| Hydrogen Peroxide | 0.017% |
| Risuteganib | 0.6-0.8%. |

22. A method according to claim 1 wherein the pharmaceutical composition comprises:

| Purified Water | q.s. to 100% |
|---|---|
| Sodium Alginate | 0.01%-15% |
| Sodium Chloride | 0.20% |
| Potassium Chloride | 0.14% |
| Magnesium Chloride | 0.011% |
| Boric Acid | 0.2% |
| Sodium Chlorite | 0.05% |
| Hydrogen Peroxide | 0.017% |
| Taurine | 0.6-0.8% |
| Risuteganib | 0.6-0.8%. |

23. A method according to claim 1 wherein the pharmaceutical composition comprises:

| Purified Water | q.s. to 100% |
|---|---|
| Sodium Alginate | 0.01%-15% |
| Sodium Chloride | 0.20% |
| Potassium Chloride | 0.14% |
| Magnesium Chloride | 0.011% |
| Boric Acid | 0.2% |
| Sodium Chlorite | 0.05% |
| Hydrogen Peroxide | 0.017% |
| Risuteganib | 0.6-0.8%. |

24. A method according to claim 1 wherein the pharmaceutical composition is administered topically to an eye of the subject.

25.-47. (canceled)
